(19) United States
(12) Patent Application Publication
(10) Pub. No.: US 2026/0199034 A1
(43) Pub. Date: Jul. 16, 2026

(54) Dual Multi-Axis Force and Torque Sensors in Robotic Surgical System

(71) Applicant: ATI Industrial Automation, Inc., Apex, NC (US)

(72) Inventors: Ian Stern, Raleigh, NC (US); Joseph Lipsey, Lillington, NC (US)

(21) Appl. No.: 19/441,465

(22) Filed: Jan. 6, 2026

Related U.S. Application Data
(60) Provisional application No. 63/743,942, filed on Jan. 10, 2025.

(51) Int. Cl. A61B 34/30; A61B 90/00

(52) U.S. Cl. CPC A61B 34/30; A61B 2034/302; A61B 2090/066

(57) ABSTRACT

A robotic system for minimally invasive surgery comprises a robot arm with a robot F/T sensor and a surgical instrument attached thereto. The surgical instrument is disposed in the body cavity of a patient through a trocar which has a trocar F/T sensor integrated therein. The trocar F/T sensor outputs are subtracted from the robot F/T sensor outputs (or vice versa), to remove the effects of forces or torques between the trocar and the patient’s skin, yielding only force or torques applied to the surgical instrument. To perform the subtraction, the reference frames of the two F/T sensors are first aligned. The position of, e.g., the tip of the surgical instrument may be measured or estimated, and the corrected forces or torques may similarly be translated to the reference frame of the surgical instrument tip.

### RELATED APPLICATIONS

This application claims the benefit of U.S. Provisional Application No. 63/743942, filed January 10, 2025, the entire disclosure of which being hereby incorporated by reference herein.

### FIELD OF DISCLOSURE

The present disclosure relates generally to robotic surgical tools, and in particular to the use of two multi-axis force and torque sensors to determine forces and/or torques applied to the end of a surgical instrument disposed in a patient’s body cavity through a trocar.

### BACKGROUND

Force and/or torque (F/T) sensors are well known in the robotic arts. F/T sensors measure forces and torques experienced by robotic arms, and provide these data to robotic control systems. F/T feedback and monitoring are critical to many robotic operations. For example, in force-controlled operations such as material removal (drilling, grinding, polishing, and the like), an F/T sensor affixed to the robot arm senses the actual force with which a robotic tool is applied to a workpiece, and the robot arm (or a compliance structure) is controlled to maintain the contact force at or below a setpoint. Examples of multi-axis F/T sensors and F/T resolution techniques include those described in U.S. Patent Nos. 10,422,707; 10,067,019; 11,892,364; 11,747,224; 11,491,663; 11,085,838; and published U.S. Patent Application No. 2023/0049155, all assigned to the assignee of the present disclosure and incorporated herein by reference in their entireties.

The use of robotics in medical and veterinary surgery has increased dramatically. Robotics enables “minimally invasive” surgical procedures, where only one or a few small incisions are made into a patient’s body and robotic tools are inserted into the body cavity. This is a dramatic difference from conventional surgery, where an opening large enough to enable a surgeon to insert his or her hands, holding hand tools, must be cut into the patient's body. A popular form of minimally invasive surgery is laparoscopic, where a light source and camera are inserted through a small incision into the body cavity. The camera sends images of the patient’s interior to a camera monitored by a surgeon. Other surgical tools, such as scalpels, forceps, and the like, are inserted through the same or different incisions, and may be directly or remotely manipulated by the surgeon to perform surgical operations inside the body cavity. Such minimally invasive surgeries may be performed manually; with the assistance of some robotic surgical tools; or fully robotically, where the surgeon controls robotic arms that physically manipulate surgical tools within the patient’s body. In the latter case, with sufficiently high-speed and reliable data communications, the surgeon can be located remotely from the patient – in the next room or in another country.

A surgeon controls the force with which he or she utilizes surgical instruments as a matter of course. Any time a surgeon’s hands are not directly controlling a surgical instrument, F/T sensors are required to ascertain these application forces, to prevent damage to tissue and ensure proper operation. For example, force and/or torque information from F/T sensors on robotic surgical instruments may be provided to a haptic feedback system of a minimally invasive surgical system. By integrating force and torque sensors into the robotic instruments, the system can relay tactile information back to the surgeon, simulating the sense of touch. This provides accurate “feel” to the actuators the surgeon uses to control the robotic surgical instruments, providing the beneficial illusion that the surgeon is holding and controlling the surgical tool directly, rather than remotely. This feedback is essential for tasks that require fine motor skills, such as suturing or dissecting tissues.

Moreover, force and torque sensing is vital for the development of advanced control algorithms in surgical robotics. These algorithms use the data from the sensors to adjust the movements of the robotic arms in real-time, ensuring smooth and precise operations. For example, in procedures such as retinal microsurgery, where even slight movements can have significant consequences, the sensors help maintain stability and control. This technology not only improves the outcomes of surgeries but also enhances the training of new surgeons by providing them with detailed feedback on their performance.

A significant challenge in incorporating F/T sensors into robotic surgical instruments is the requirement of miniaturization of the F/T sensors. Surgical instruments, especially those used in minimally invasive procedures, need to be very small to fit through tiny incisions. Integrating sensors that can accurately measure force and torque without compromising the instrument’s size and functionality is a challenge.

Another challenge is ensuring the durability and reliability of these sensors in the demanding environment of surgery. Surgical instruments must withstand sterilization processes, which often involve high temperatures and harsh chemicals. The sensors must be robust enough to maintain their accuracy and functionality after repeated sterilizations. Additionally, they need to be resistant to bodily fluids and other contaminants encountered during surgery.

A trocar is a medical instrument that is critical to minimally invasive surgery. A trocar may include an obturator, a cannula, and a seal. The obturator is a pointed or blunt instrument that facilitates the initial penetration into the body cavity. The cannula is a hollow tube that remains in place after the obturator is removed, providing a pathway for other surgical instruments to be inserted. The seal ensures that no air or fluids escape from the body cavity during the procedure. In laparoscopic surgery, a trocar allows a surgeon to introduce cameras and other instruments into the patient’s abdominal cavity without making large incisions, significantly reducing recovery time and the risk of complications.

It is known to integrate a force sensing system into a housing of a trocar or an inserted obturator. Such force sensing systems detects one force in one direction – along the longitudinal axis of the trocar/cannula and towards the patient. For example, the force sensing system may emit one indication (e.g., illuminates a green LED) so long as the applied force is below a predetermined threshold, and a different indication (e.g., illuminates a red LED) if the applied force exceeds the predetermined threshold. This type of force sensing system addresses the situation when a clinician may attempt to use a surgical tool (e.g., a stapler, forceps, etc.) at a position within the body cavity that is further from the incision than the length of the tool.

It is further known to attach force sensing elements, such as strain gages, to an outer sleeve of a cannula near the point of insertion into a body wall. The strain gages detect and measure force applied to the outer wall of the outer tube, by contact with the body wall, in a direction generally transverse to the longitudinal dimension of the cannula. Because the outer wall is not mechanically connected to the cannula, the strain gages are isolated from instrument-cannula interactions as instruments are manipulated within the cannula.

U.S. provisional patent application serial no. 63/742,115, titled Trocar with Multi-Axis Force and Torque Sensing, filed Jan. 06, 2025, and assigned to the assignee of the present disclosure, describes the integration of a multi-axis F/T sensor on the cannula of a trocar – referred to herein as a trocar F/T sensor. The disclosure of this application is incorporated herein by reference, in its entirety.

Neither a conventional F/T sensor on a robot arm, nor a trocar F/T sensor as known in the prior art, provides data reflecting forces and torques actually experienced by the end, or tip, of a robotic surgical instrument. Both are disposed outside the patient’s body, and may register forces, e.g., between the trocar and patient’s skin, which are not generated by contact between the robotic surgical instrument and tissue within the patient’s body cavity, which are the F/T measurements of interest. Integrating a F/T sensor directly on the surgical instrument is problematic, due to the size and reliability challenges discussed above, as well as replicating the cost of F/T sensor instrumentation across every surgical instrument. Accordingly, ascertaining and reporting actual forces and torques experienced at the distal end of a surgical instrument stands as a significant challenge in advancing the state of the art of minimally invasive surgery.

The Background section of this document is provided to place aspects of the present disclosure in technological and operational context, to assist those of skill in the art in understanding their scope and utility. Approaches described in the Background section could be pursued, but are not necessarily approaches that have been previously conceived or pursued. Unless explicitly identified as such, no statement herein is admitted to be prior art merely by its inclusion in the Background section.

### SUMMARY

The following presents a simplified summary of the disclosure in order to provide a basic understanding to those of skill in the art. This summary is not an extensive overview of the disclosure and is not intended to identify key/critical elements of aspects of the disclosure or to delineate the scope of the disclosure. The sole purpose of this summary is to present some concepts disclosed herein in a simplified form as a prelude to the more detailed description that is presented later.

According to one or more aspects described and claimed herein, a robotic system for minimally invasive surgery comprises a robot arm with a robot F/T sensor and a surgical instrument attached thereto. The surgical instrument is disposed in the body cavity of a patient through a trocar which has a trocar F/T sensor integrated therein. The trocar F/T sensor outputs are subtracted from the robot F/T sensor outputs (or vice versa), to remove the effects of forces and/or torques between the trocar and the patient’s skin, yielding only force and/or torques applied to the surgical instrument. To perform the subtraction, the reference frames of the two F/T sensors are first aligned. The position of, e.g., the tip of the surgical instrument may be measured or estimated, and the corrected forces and/or torques may similarly be translated to the reference frame of the surgical instrument tip.

One embodiment relates to a dual, multi-axis, force and/or torque (F/T) sensor robotic system for minimally invasive, robotic, and/or robotic-assisted surgery. The robotic system includes a multi-axis robot F/T sensor attached to a robotic arm; a multi-axis trocar F/T sensor integrated within a surgical trocar; and a robotic surgical instrument attached to the robot arm and disposed in a body cavity of a surgical patient through the trocar. The robotic system further includes processing circuitry configured to receive outputs from the robot F/T sensor and trocar F/T sensor. The processing circuitry is further configured to translate and/or rotate the reference frame of one of the robot F/T sensor and the trocar F/T sensor to the reference frame of the other of the robot F/T sensor and the trocar F/T sensor; subtract forces and/or torques from the one of the F/T sensors from the other of the F/T sensors; and output the resulting forces and/or torques applied to the surgical instrument.

Another embodiment relates to a method of performing minimally invasive surgery using a robotic surgical instrument attached to a robotic arm and disposed in a body cavity of a patient through a trocar. Forces and/or torques are sensed at the robotic arm in a first reference frame. Forces and/or torques are sensed at the trocar in a second reference frame. One of the first and second reference frames is translated and/or rotated to the other of the first and second reference frames. Forces and/or torques sensed in the one of the first and second reference frames are subtracted from the forces and/or torques sensed in the other of the first and second reference frames. The resulting forces and/or torques are output.

### BRIEF DESCRIPTION OF THE DRAWINGS

The present disclosure will now be described more fully hereinafter with reference to the accompanying drawings, in which aspects of the disclosure are shown. However, this disclosure should not be construed as limited to the aspects set forth herein. Rather, these aspects are provided so that this disclosure will be thorough and complete, and will fully convey the scope of the disclosure to those skilled in the art. Like numbers refer to like elements throughout.

FIG. 1 is a plan view of a prior art robotic force/torque sensor.

FIG. 2 is an enlarged and exaggerated view of a deformable beam of the F/T sensor of FIG. 1 under an applied force.

FIG. 3 is a perspective view of a cannula of a trocar for robotic surgery.

FIG. 4 is a top plan view of the cannula of FIG. 3.

FIG. 5 is an isometric cross section view of the cannula of FIGS. 3 and 4.

FIG. 6 is a side sectional view of a second cannula.

FIG. 7 is an isometric sectional view of the cannula of FIG. 6.

FIG. 8 is a side view of the cannula of FIGS. 6 and 7.

FIG. 9 is a top plan view of the cannula of FIGS. 3-5, with transducers and a controller.

FIG. 10 shows matrix calculations for the rotation and translation of forces in torques between reference frames.

FIG. 11 is a perspective view showing three coordinate systems on a robotic surgical instrument assembly.

FIG. 12A is a sectional view showing misalignment of an unloaded surgical instrument in a cannula.

FIG. 12B is a sectional view showing misalignment of a loaded (bent) surgical instrument in a cannula.

FIG. 13 is a flow diagram of a method of performing minimally invasive surgery using a robotic surgical instrument attached to a robotic arm and disposed in a body cavity of a patient through a trocar.

### DETAILED DESCRIPTION

For simplicity and illustrative purposes, the present disclosure is described by referring mainly to an exemplary aspect thereof. In the following description, numerous specific details are set forth in order to provide a thorough understanding of the present disclosure. However, it will be readily apparent to one of ordinary skill in the art that the present disclosure may be practiced without limitation to these specific details. In this description, well known methods and structures have not been described in detail so as not to unnecessarily obscure the present disclosure.

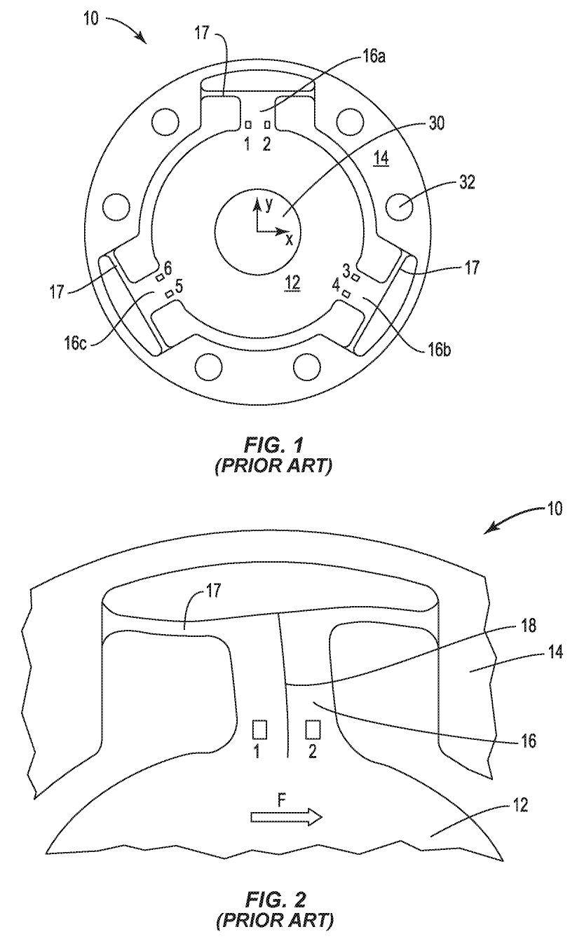

One approach to F/T sensor design is briefly reviewed, with reference to U.S. patent No. 10,422,707, incorporated above. FIG. 1, which is a reproduction of FIG. 1 of the ‘707 patent, shows a plan view of a force/torque sensor 10. A rigid hub 12 is connected to a rigid annular ring 14 by three deformable beams 16a, 16b, 16c. In the embodiment depicted, each beam 16 connects directly to the hub 12, and connects to the annular ring 14 by flexures 17, which aid in the deformation of the beams 16 under mechanical loading.

Affixed to the upper surface of each deformable beam 16 are strain gages 1-6. FIG. 1 also depicts two axes of a 3-dimensional reference Cartesian coordinate system (z-direction extending out of the figure), to which resolved forces and torques are referenced.

FIG. 2, which is a reproduction of FIG. 2 of the same ‘707 patent, is an enlarged view of one beam 16a, showing an exaggerated deformation due to a force F applied to the hub 12, relative to the annular ring 14. This force deforms the beam 16a slightly to the left (the figure is not to scale). A compressive force is induced on the left side surface of deformable beam 16a, and a tensile force is induced on the right side surface. Transducers, such as strain gages, may be mounted on these surfaces, where they generate strong signals, of opposite polarity, from which the deformation, and hence the applied force F, could be ascertained. In embodiments of the ‘707 patent, the strain gages 1-6 are mounted only on an upper surface of the deformable beams 16, spaced apart from a neutral axis 18. The ‘707 patent, as well as others of the patents incorporated above, describes the signal processing by which multi-axis forces and/or torques between the hub 12 and annular ring 14 are resolved and quantified.

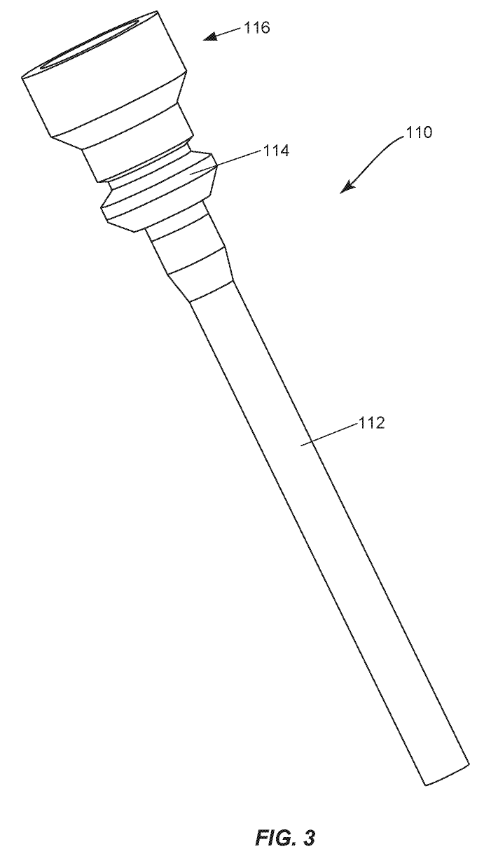

FIG. 3 depicts a perspective view of a cannula 110 for a trocar for minimally invasive surgery. The cannula 110 includes an elongated tube 112, which during minimally invasive surgery is inserted into a patient’s body cavity through a small incision. Surgical tools are inserted into the body cavity through the tube 112 of the cannula 110. An inner seal 114 is positioned within the patient’s body, just below the skin. The inner seal 114 prevents gas or fluid from escaping through the incision during surgical operations. A trocar F/T sensor 116 is disposed on the end of the cannula 110, opposite the inner seal 114.

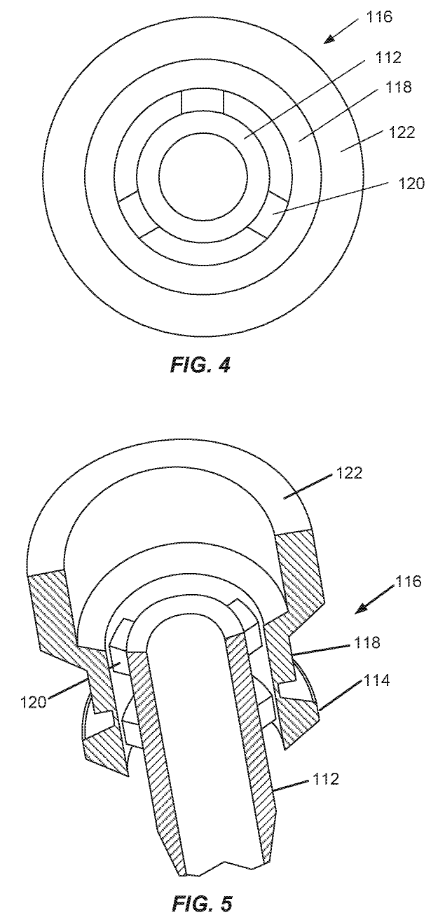

FIGS. 4 and 5 show details of the multi-axis F/T sensor 116. An annular sleeve 118 extends around the tube 112, at least partially along its length. The tube 112 and annular sleeve 118 are relatively rigid, and are connected at a plurality of radial positions – in some embodiments, resembling the spokes on a wheel – by deformable members 120. The deformable members 120 deform slightly in response to forces and/or torques between the tube 112 and annular sleeve 118. Transducers (not shown) affixed to one or more surfaces of a plurality of deformable members 120 sense tensile and/or compressive forces induced on surfaces of the deformable members 120 by the applied forces and/or torques, and generate outputs, such as electrical signals. These are transmitted, such as by wires (not shown) to a controller (not shown), which resolves the sensed tensile and compressive forces into forces and/or torques along a plurality of axes. In one embodiment, an extended annular member 122 provides space for an inner seal or other components to be fitted onto the cannula 110.

As best seen in FIG. 5, in one embodiment the deformable members 120 are formed in two sets of three, which sets are laterally spaced apart from each other along the length of the tube 112. In various embodiments, any number of deformable members 120 in each set may be radially spaced around the tube 112, and there may be more than two sets of deformable members 120 spaced along the tube 112.

Regardless of the specific configuration of deformable members 120, the multi-axis F/T sensor 116 is configured to sense and resolve forces and/or torques between the tube 112 and the annular sleeve 118. In a surgical operation, the annular sleeve 118 is held in place on the patient’s body, and surgical instrument are inserted through the tube 112. Forces and/or torques between the tube 112 and annular sleeve 118, such as those induced by a surgeon and/or robot manipulating a surgical tool disposed in the tube 112, are sensed, resolved, and reported (and/or fed back to a haptic feedback system) by the multi-axis F/T sensor 116.

In general, enough transducers are attached to the deformable members 120 to resolve between three and six axes of forces and torques. At least one deformable member 120 is required per resolved axis; however, each deformable member 120 may have multiple transducers affixed thereto.

In the embodiment shown in FIGS. 4 and 5, there are two sets of three deformable members 120 connecting the tube 112 and annular sleeve 118. At least two deformable members 120 are required. In the depicted embodiment there are six total deformable members 120 for strength purposes; however, a typical embodiment may include only three or four. The deformable members 120 may be more complicated than the simple beams depicted. For example, they may be L-shaped or T-shaped, and/or may include flexures as shown in FIGS. 1 and 2 and described in the ‘707 patent. The deformable members 120 may also be parallel or tangential to the tube 112 instead of radial, as shown.

In the embodiment depicted in FIG. 5, the annular sleeve 118 extends along the length of the tube 112 only for a distance sufficient to encompass all (e.g., both sets of) deformable members 120. In another embodiment (not shown), the annular sleeve 118 extends along the entire length of the tube 112. This isolates the tube 112 from forces that are seen by external factors that are unrelated to force applied to the tube 112. However, it requires a larger incision, as the outer diameter of the cannula 110 is now larger.

As described in the above incorporated patents, the transducers affixed to the deformable members 120 may comprise strain gages, such as silicon strain gages. However, the invention is not limited to strain gages. In other embodiments, the transducers may comprise capacitive, fiber bragg grating, Surface Acoustic Wave (SAW), or piezoelectric sensors, or combinations thereof.

The cannula 110 may be formed as a single-piece system, or alternatively may comprise two or more modular pieces that are assembled into an operative cannula 110.

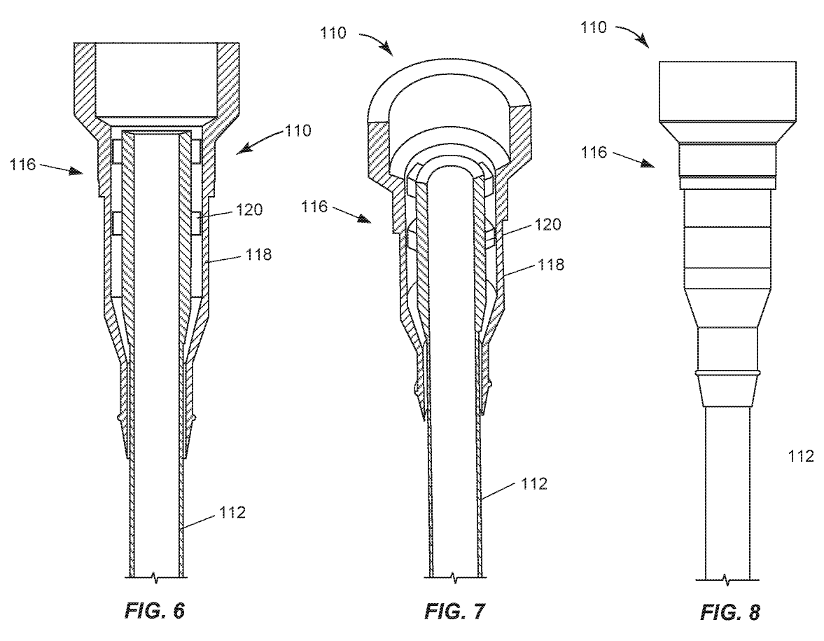

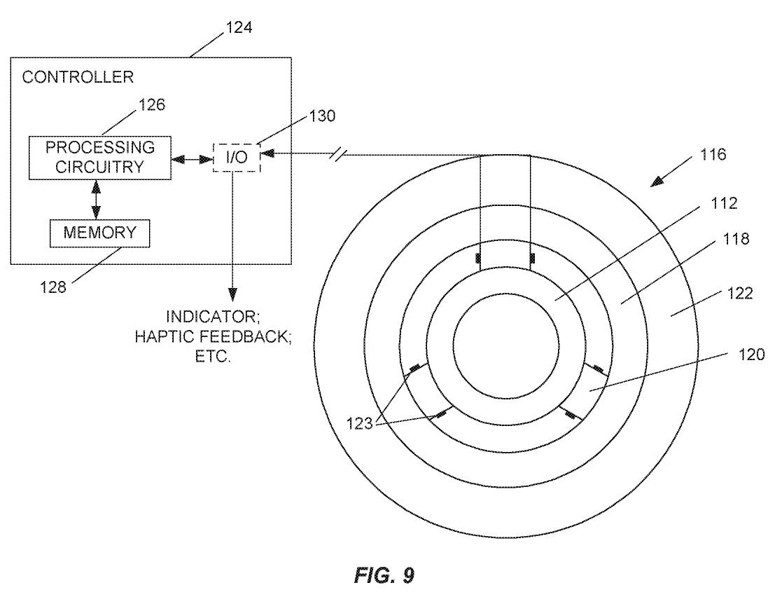

FIGS. 6, 7, and 8 show a slightly different cannula 110, also with a trocar F/T sensor 116 integrated therein. An annular sleeve 118 extends around the tube 112, at least partially along its length. The tube 112 and annular sleeve 118 are relatively rigid, and are connected at a plurality of radial positions – in some embodiments, resembling the spokes on a wheel – by deformable members 120. The deformable members 120 deform slightly in response to forces and/or torques between the tube 112 and annular sleeve 118.

FIG. 9 depicts the cannula 110 of FIG. 3, showing transducers 123, in this embodiment strain gages, connected to the side walls of deformable members 120. In other embodiments, other transducers 123 may be used, and/or they may be attached to the deformable members 120 in different locations. In one embodiment, one or more transducers 123 may be attached to the tube 112 or annular sleeve 118 proximate to a deformable member 120.

The transducers 123 are operatively connected to a controller 124 (only one pair shown connected in FIG. 9). The controller 124 includes processing circuitry 126 operatively connected to memory 128, and optionally (as indicated by dashed lines) input/output (I/O) circuitry 130. The controller 124 may be integrated into the cannula 110 or trocar, or may be part of a laparoscopic or robotic surgical system.

The processing circuitry 126 is configured to receive electrical signals from transducers 123, and to resolve the signals into forces and/or torques applied between the annular sleeve 118 and the tube 112 along at least three axes. Algorithms for resolving transducer outputs into forces and torques are known in the art, and are disclosed, for example, in the above incorporated patents. These algorithms are embodied in machine-readable code stored in memory 128, which is accessed by the processing circuitry 126.

Optional I/O circuitry may pre-process the transducer 123 output signals prior to their processing by the processing circuitry 126, such as by amplifying, low-pass filtering to reduce noise, and performing analog to digital conversion. The I/O circuity may also post-process the resolved forces and torques, such as by comparing them to predetermined thresholds to emit an alarm if excessive force or torque is detected, by transforming the F/T to a format required by haptic feedback circuitry in a surgical controller, or for other uses. Alternatively, the processing circuitry 126 may perform all formatting of the output data.

The processing circuitry 126 may comprise any sequential state machine operative to execute machine instructions stored as machine-readable computer programs in memory 128, such as one or more hardware-implemented state machines (e.g., in discrete logic, FPGA, ASIC, etc.), programmable logic together with appropriate firmware; one or more stored-program, general-purpose processors, such as a microprocessor or Digital Signal Processor (DSP), together with appropriate software; or any combination of the above.

The memory 128 may comprise any non-transitory machine-readable media known in the art or that may be developed, including but not limited to magnetic media (e.g., floppy disc, hard disc drive, etc.), optical media (e.g., CD-ROM, DVD-ROM, etc.), solid state media (e.g., SRAM, DRAM, DDRAM, ROM, PROM, EPROM, Flash memory, solid state disc, etc.), or the like.

The optional I/O circuitry 126 may comprise receiver circuitry configured to receive, amplify, and digitize electrical signals from transducers 123. The I/O circuitry may additionally include transmitter or interface circuitry configured to output useful force/torque data to other systems or components, such as an excessive force indicator (e.g., flashing light, audible alarm, or the like), as data for a haptic feedback system, or the like.

The trocar F/T sensor 116 is configured to sense and resolve forces and/or torques between the tube 112 and the annular sleeve 118. In a surgical operation, the annular sleeve 118 is held in place on the patient’s body, and surgical instrument are inserted through the tube 112. Forces and/or torques between the tube 112 and annular sleeve 118, such as those induced by a surgeon and/or robot manipulating a surgical tool disposed in the tube 112, are sensed, resolved, and reported (or fed back to a haptic feedback system) by the trocar F/T sensor 116.

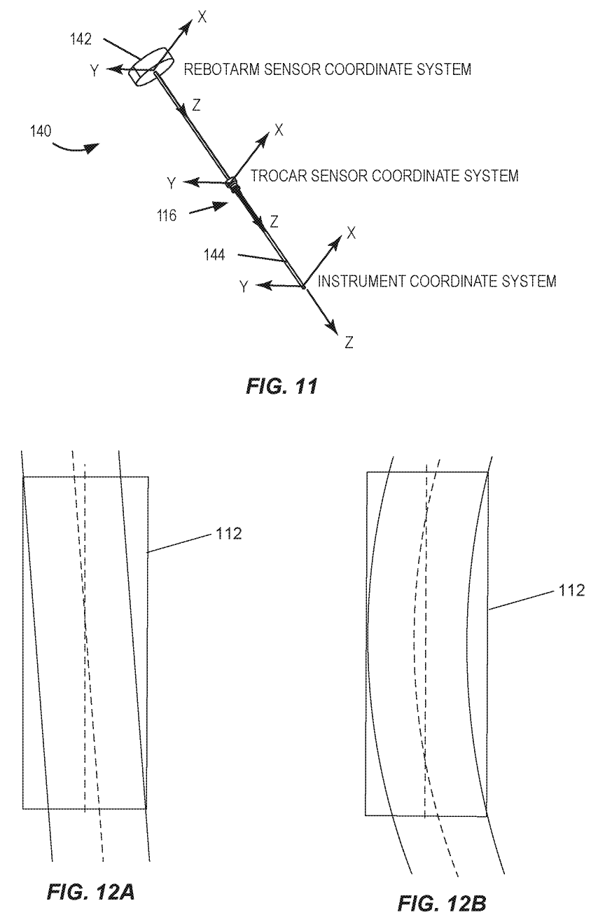

While the trocar F/T sensor 116 is an improvement over a conventional robotic F/T sensor 142 attached to a robotic arm (see FIG. 11), it still does not directly measure forces or torques at the tip of the surgical instrument. A single F/T sensor, whether mounted on the robot arm or integrated into a trocar, measures a superposition of forces and torques experienced by the surgical instrument, and those between the trocar and the patient’s skin. Although best surgical practices strive to minimize the latter, they are always present to some extent, and they distort the sensed forces and torques of the surgical instrument.

According to embodiments of the present disclosure, forces and/or torques applied to the tip of a robotic surgical instrument are deduced by sensing both robot arm forces and/or torques, and trocar forces and/or torques, and subtracting one from the other. This process is described herein for the case of subtracting trocar forces and/or torques from the robot arm forces and/or torques; however, the operation could be the reverse and will still yield only the surgical instrument forces and/or torques.

FIG. 11 shows a conventional F/T sensor mounted on a robot arm (referred to herein as the robot F/T sensor 142), and a robotic surgical instrument is attached to the robot arm. If the surgical instrument were operating on a patient’s skin, the robot F/T sensor 142 would be sufficient to ascertain forces and/or torques experienced by the surgical instrument, for example, to be fed back to a haptic feedback system. However, when the surgical instrument is inserted into the interior of a patient’s body cavity through a trocar 110 disposed in an incision in the patient’s skin, at least some forces and/or torques are generated between the trocar 110 and the skin. These are sensed by a trocar F/T sensor 116, and subtracted from the forces and/or torques sensed by the robot F/T sensor 142, yielding the forces and/or torques experienced by the surgical instrument.

The forces and/or torques subtracted are fully resolved. Some F/T sensors output only transducer outputs, which are generated in the reference frame of the F/T sensor. These signals must be processed to resolve them into forces and/or torques. Numerous techniques are known in the art to accomplish this, some of which are detailed in the patents incorporated herein above. Some F/T sensors include processing circuitry that performs these calculations, and the sensors output resolved forces and/or torques. Some F/T sensors may also include accelerometers, Inertial Measurement Units (IMU), or other circuitry that outputs useful information. For the purpose of discussion herein, it is assumed both the robot F/T sensor 142 and the trocar F/T sensor 116 output fully resolved forces and/or torques, each referenced to the respective sensor’s local reference frame or coordinate system. For F/T sensors that output only transducer signals, external signal processing will be required, which is within the skill of those of skill in the art, and is not further elaborated herein.

To subtract the trocar F/T sensor 116 outputs from the robot F/T sensor 142 outputs, the reference frame of the former must first be aligned to the reference frame of the latter. At a minimum, this requires a translation of the trocar F/T sensor 116 reference frame to the position of the robot F/T sensor 142 reference frame, and in general, may require a rotation of one or more axes.

FIG. 11 shows a robotic surgical instrument assembly 140 and three frames of reference, or coordinate systems: one at the robot F/T sensor 142, one at the trocar F/T sensor 116, and one at the tip of the surgical instrument 144. In this example, only a translation is required; however, in general a joint or pivot point may allow a surgical instrument to assume an orientation other than along a longitudinal axis of the robot arm. In this case, a rotation of one or more axes is also required.

The reference frames for the two sensors 116, 142 can be found, e.g., through accelerometer, IMU, magnetometer, GPS data, capacitance measurements using the tool, linear encoders that interface between the tool and trocar, or various other means. Additionally, on the robot side, the reference frame can be found from encoders on the robot arm joints that can be resolved into a final position. This can then be used to determine the reference frame of the trocar 110, using methods such as vision or laser measurement. It can also be approximated by mathematical means to get an idea of how far away the two are from each other by looking at the resolved forces and torques of both sensors 116, 142.

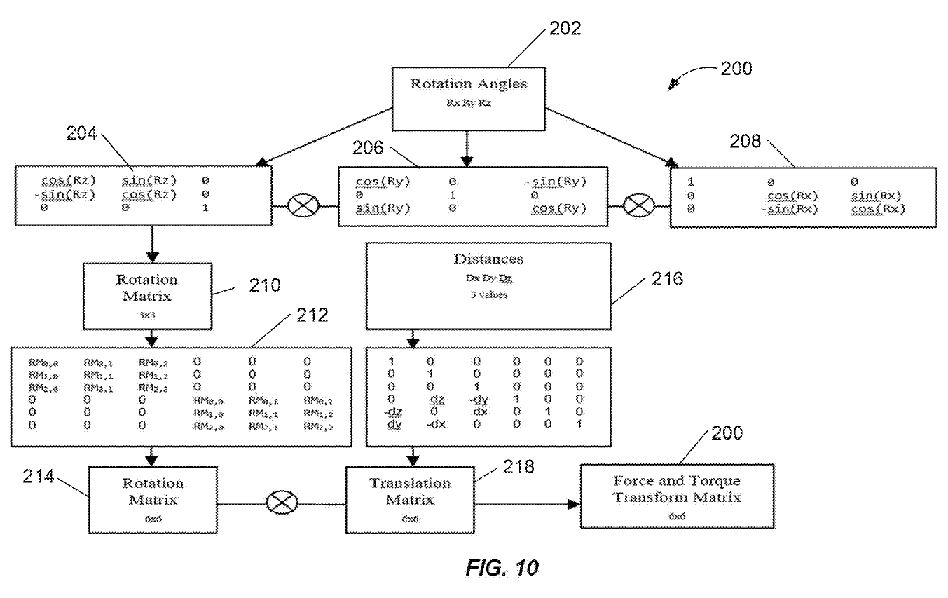

Once the angles between the two reference frames are found, along with the distances in all axes, the computations 200 shown in FIG. 10 are completed in order to place forces and torques from both sensors in the same reference frame. FIG. 10 shows one procedure for calculating a Force and Torque Transform Matrix 220 to transform outputs of the trocar F/T sensor 116 to the frame of reference of the robot F/T sensor 142 (or vice versa). Block 202 indicates rotation angles Rx, Ry, Rz for the three axes. These are detailed in the Rz matrix 204, the Ry matrix 206, and the Rx matrix 208. These three are multiplied together to yield a 3x3 Rotation Matrix 210. The 3x3 Rotation matrix 210 is then expanded, by duplication and padding, into a 6x6Rotation Matrix 214, the contents of which are shown at block 212. Block 216 indicates translation distances Dx, Dy, Dz, which are entered into a 6x6 Translation Matrix 220, the contents of which are shown at block 218. The 6x6 Rotation Matrix 214 and the6x6 Translation Matrix 220 are multiplied, yielding a 6x6 Force and Torque Transform Matrix 222.

Force and/or torque outputs of the trocar F/T sensor 116 are multiplied by the Force and Torque Transform Matrix 222, translating them to the reference frame of the robot F/T sensor 142. The translated trocar F/T sensor 116 outputs are then subtracted from the corresponding robot F/T sensor 142 outputs. For F/T sensors 116, 142 that output fewer than 6 axes of data, the missing axes are simply omitted from the rotation/translation calculations. The results are the projected forces and/or torques acting on the robotic surgical instrument attached to the robot arm and disposed in a patient’s body cavity through the trocar 110. These force and/or torque values may be used in a haptic feedback mechanism of a robotic surgical system, used by automated procedures, stored and used for training of surgeons and/or Machine Learning models, and the like.

In some applications, it is important, or at least useful, to know the location of a particular point on the surgical instrument, such as the tip. When the position of the tip of the instrument is known or can be calculated with high precision, a Force and Torque Transform Matrix 222 as described above is used to rotate and/or translate forces and/or torques sensed by the robot F/T sensor 142 (compensated for those sensed by the trocar F/T sensor 116) to the desired position, such as the surgical instrument tip. The position of the tip may be ascertained, for example, by vision, magnetometer, GPS, inductive sensors built into the trocar, IMU data, an accelerometer, or the like.

Surgical instruments are designed to be thin for several reasons. Thin instruments allow for greater precision and control. The slender design also minimizes tissue damage and reduces the risk of infection by limiting the size of incisions. Additionally, thin tools can navigate through narrow and complex anatomical structures more easily, enhancing the surgeon’s ability to perform minimally invasive surgeries.

Due to thin design, and consequent dearth of material in surgical instruments, they tend to exhibit noticeable deflections. These deflections will cause a change in the coordinate system at the end of the tool, which would cause a change in the values needed for the tooling transformation. This change may be ignored for low precision applications, but for other applications it must be measured or estimated.

In one embodiment, the position of the desired point on the surgical instrument (e.g., the tip) is determined mathematically. With a known stiffness of the surgical tool, the tip location is calculated by knowing the approximate direction of the load applied to the tip. The directional angle difference between the Z- axis of the robot F/T sensor 142 and trocar F/T sensor 116 in the reference frames must also be known. FIG. 11 shows the relevant reference frames.

If there is an angle difference between the two Z axes that mathematically confirms that the cannula is in contact with the surgical instrument, there are two possible states. FIG. 12A depicts one, where the instrument not loaded or bent, and is simply “canted” within the cannula. FIG. 12B depicts the state where the instrument is loaded and deflected. The view of FIG. 12B is with a load that is applied to the instrument with a force vector that is pointing towards the direction that the tool is coming from. This won’t always be the case, and the output vector of the distal end of the tool will change accordingly. The views of 12A and 12B are simplified, and presented only to describe the possibilities.

By knowing the direction and magnitude of forces applied to the tip of the surgical instrument, the location of the tip is determined mathematically, by a variety of methods.

In one embodiment, when the tip is in an unloaded state, the size difference between the cannula internal width and the width of the surgical tool, in combination with the angle mismatch and known Z distance, are used to determine the approximate placement of the tip of the instrument in relation to the two F/T sensors 116, 142.

If the tip is in a loaded state, a simple vector is calculated based on the theoretical direction leaving the trocar. An additional transformation is optionally completed based on the stiffness of the tool, to account for tooling deflections after leaving the trocar. Alternatively, a simplified finite element analysis may be completed to quantify the amount of deflection.

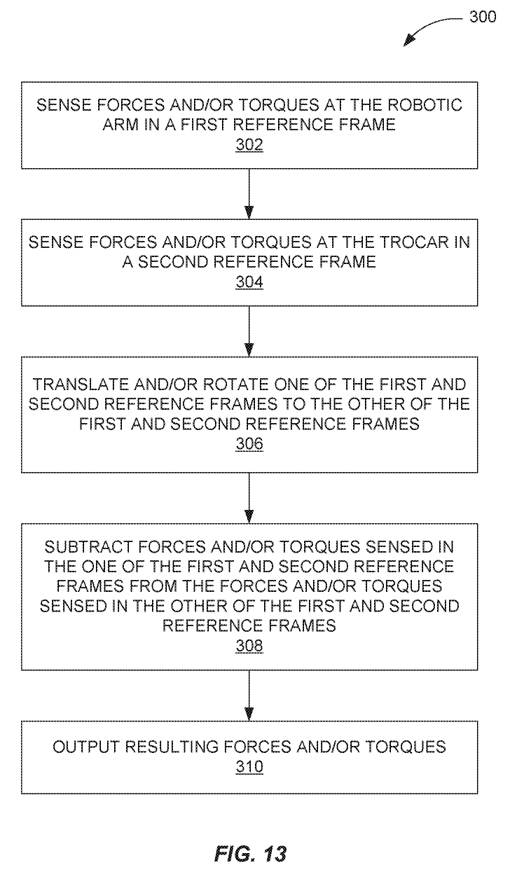

FIG. 13 depicts the steps in a method 300 of performing minimally invasive surgery using a robotic surgical instrument attached to a robotic arm and disposed in a body cavity of a patient through a trocar. Forces and/or torques are sensed at the robotic arm in a first reference frame (block 302). Forces and/or torques are sensed at the trocar in a second reference frame (block 304). One of the first and second reference frames is translated and/or rotated to the other of the first and second reference frames (block 306). Forces and/or torques sensed in the one of the first and second reference frames are subtracted from the forces and/or torques sensed in the other of the first and second reference frames (block 308). The resulting forces and/or torques are output (block 310).

Embodiments of the present disclosure present significant advantages over the prior art, and may achieve one or more of the following technical effects. Due to the challenging environment of preparation for, and use in, surgery, surgical instruments must be highly rugged and reliable. Also, the instruments are as thin as possible, to minimize the size of incisions in the patient’s body. Due to these constraints, is it extremely difficult to position an F/T sensor directly at the point of desired force/torque sensing, which is usually the tip of the surgical instrument. A single F/T sensor mounted elsewhere along the robotic stack will not yield accurate force/torque readings, as it includes both surgical instrument and trocar/body forces and torques. By using two multi-axis F/T sensors 116, 142, and subtracting trocar 110 forces from the robotic F/T sensor 142 outputs (or vice versa), forces and/or torques applied to the surgical instrument itself are more accurately estimated. Both the robot F/T sensor 142 and trocar F/T sensor 116 are outside the body, where there is more room to build deformable structures. Also, a wide variety of surgical tools may be used with the same robot arm and trocar 110, so the cost of the F/T sensors 116, 142 is amortized, and need not be separately incurred for every surgical instrument.

Generally, all terms used herein are to be interpreted according to their ordinary meaning in the relevant technical field, unless a different meaning is clearly given and/or is implied from the context in which it is used. All references to a/an/the element, apparatus, component, means, step, etc. are to be interpreted openly as referring to at least one instance of the element, apparatus, component, means, step, etc., unless explicitly stated otherwise. Any feature of any of the aspects disclosed herein may be applied to any other aspect, wherever appropriate. Likewise, any advantage of any of the aspects may apply to any other aspects, and vice versa. Other objectives, features, and advantages of the enclosed aspects will be apparent from the description.

As used herein, the term “configured to” means set up, organized, adapted, or arranged to operate in a particular way; the term is synonymous with “designed to,” or in the case of processing circuitry and/or software, “programmed to.” As used herein, a “surgical tool” is any device that may be inserted into a patient’s body cavity during minimally-invasive surgery, whether it performs a specific surgical function or supports other surgical tools. For example, a laparoscopic camera is a surgical “tool,” even though it does not directly interact with the patient’s body. As used herein, the coordinating conjunction “or” has the meaning of the Boolean logical operator OR – for example, “A or B” is true if A is true, B is true, or both A and B are true; it is false only if both A and B are false. Hence, the term “or” subsumes the common phrase “and/or.”

The present disclosure may, of course, be carried out in other ways than those specifically set forth herein without departing from essential characteristics of the disclosure. The present aspects are to be considered in all respects as illustrative and not restrictive, and all changes coming within the meaning and equivalency range of the appended claims are intended to be embraced therein.

1. A dual, multi-axis, force or torque (F/T) sensor robotic system for minimally invasive, robotic, or robotic-assisted surgery, comprising:
a multi-axis robot F/T sensor attached to a robotic arm;
a multi-axis trocar F/T sensor integrated within a surgical trocar; and
a robotic surgical instrument attached to the robot arm and disposed in a body cavity of a surgical patient through the trocar; and
processing circuitry configured to receive outputs from the robot F/T sensor and trocar F/T sensor and further configured to
translate or rotate the reference frame of one of the robot F/T sensor and the trocar F/T sensor to the reference frame of the other of the robot F/T sensor and the trocar F/T sensor;
subtract forces or torques from the one of the F/T sensors from the other of the F/T sensors; and
output the resulting forces or torques applied to the surgical instrument.

2. The robotic system of claim 1, wherein one or both of the F/T sensors outputs, to the processing circuitry, resolved forces or torques with respect to its reference frame.

3. The robotic system of claim 1, wherein one of the F/T sensors outputs, to the processing circuitry, signals from transducers in the F/T sensor, and wherein the processing circuitry is further configured to resolve the transducer signals into forces or torques in the frame of the F/T sensor.

4. The robotic system of claim 1, wherein the processing circuitry is further configured to:
calculate a position of a predetermined portion of the robotic surgical instrument; and
translate or rotate the reference frame from the one of the F/T sensors to the position of the predetermined portion of the robotic surgical instrument.

5. The robotic system of claim 1, wherein the surgical trocar comprises a cannula comprising a tube configured to be inserted into the body cavity of a patient, and wherein the multi-axis trocar F/T sensor integrated within the surgical trocar comprises:
an annular sleeve in spaced annular relationship to the tube; and
at least two instrumented, deformable members connecting the tube and annular sleeve;
wherein the multi-axis trocar F/T sensor is configured to sense and resolve forces or torques applied between the annular sleeve and the tube along at least three axes.

6. A method of performing minimally invasive surgery using a robotic surgical instrument attached to a robotic arm and disposed in a body cavity of a patient through a trocar, the method comprising:
sensing forces or torques at a first location in a first reference frame;
sensing forces or torques at a second location in a second reference frame;
translating or rotating forces or torques sensed in the second reference frame to the first reference frame;
subtracting the translated or rotated forces or torques sensed in the second reference frame from the forces or torques sensed in the first reference frame; and
outputting resulting forces or torques.

7. The method of claim 6 wherein sensing forces or torques comprises receiving signals from transducers in a force/torque (F/T) sensor, and further comprising resolving the transducer signals into forces or torques in the reference frame of the F/T sensor.

8. The method of claim 6 wherein translating or rotating forces or torques sensed in the second reference frame to the first reference frame comprises:
determining a 3x3 rotation angle matrix for each of three orthogonal directions required to rotationally align the second reference frame with the first reference frame;
multiplying the three 3x3 rotation angle matrices to yield a 3x3 rotation matrix;
expanding the 3x3 rotation matrix into a 6x6 rotation matrix by duplication and padding;
determining distances in each of three orthogonal directions required to translate the second reference frame to align with the first reference frame, and entering the distances into a 6x6 translation matrix;
multiplying the 6x6 rotation matrix and the 6x6 translation matrix to yield a 6x6 force and torque transform matrix; and
multiplying force or torque measurements sensed in the second reference frame by the 6x6 force and torque transform matrix.

9. The method of claim 6 wherein the first reference frame is centered at a multi-axis robot F/T sensor attached to the robotic arm and the second reference frame is centered at a multi-axis trocar F/T sensor integrated within the trocar.

10. The method of claim 6 wherein the second reference frame is centered at a multi-axis robot F/T sensor attached to the robotic arm and the first reference frame is centered at a multi-axis trocar F/T sensor integrated within the trocar.

11. The method of claim 6 further comprising:
ascertaining a position of a predetermined portion of the robotic surgical instrument; and
translating or rotate the reference frame from the one of the first and second reference frames to the position of the predetermined portion of the robotic surgical instrument.
